(19) United States
(12) Patent Application Publication
(10) Pub. No.: US 2007/0265273 A1
(43) Pub. Date: Nov. 15, 2007

(54) Pyrimidin-2-Amine Derivatives and Their Use as A2b Adenosine Receptor Antagonists

(76) Inventors: Bernat Vidal Juan, St. Cebria de Vallalta (ES); Cristina Esteve Trias, St. Pere de Riudebitlles (ES)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 10/574,101

(22) PCT Filed: Sep. 22, 2004
(86) PCT No.: PCT/EP04/10644

§ 371 Date: Mar. 8, 2007

(30) Foreign Application Priority Data
Oct. 2, 2003 (ES) P200302275

(51) Int. Cl. A61K 31/506; A61K 31/501; A61P 1/00; A61P 11/06; A61P 11/08; A61P 29/00; A61P 3/10; A61P 37/08; A61P 9/10; C07D 237/00; C07D 239/42

(52) U.S. Cl. 514/252.02; 514/274; 514/275; 544/238; 544/296

(57) ABSTRACT

This invention is directed to new potent and selective antagonists of the A_{2B} adenosine receptor having the general formula (I); to processes for their preparation; to pharmaceutical compositions comprising them; and to their use in therapy.

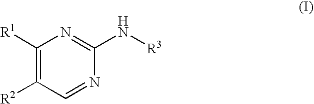

The present invention relates to new antagonists of the A_{2B} adenosine receptor. These compounds are useful in the treatment, prevention or suppression of diseases and disorders known to be susceptible of being improved by antagonism of the A_{2B} adenosine receptor, such as asthma, allergic diseases, inflammation, atherosclerosis, hypertension, gastrointestinal tract disorders, cell proliferation disorders, diabetes mellitus and autoimmune diseases.

Adenosine regulates several physiological functions through specific cell membrane receptors, which are members of the G-protein coupled receptor family. Four distinct adenosine receptors have been identified and classified: A₁, A_{2A}, A_{2B} and A₃.

The A_{2B} adenosine receptor subtype (see Feoktistov, I., Biaggioni, I. Pharmacol. Rev. 1997, 49, 381-402) has been identified in a variety of human and murine tissues and is involved in the regulation of vascular tone, smooth muscle growth, angiogenesis, hepatic glucose production, bowel movement, intestinal secretion, and mast cell degranulation.

In view of the physiological effects mediated by adenosine receptor activation, several A_{2B}receptor antagonists have been recently disclosed for the treatment or prevention of, asthma, bronchoconstriction, allergic diseases, hypertension, atherosclerosis, reperfusion injury, myocardial ischemia, retinopathy, inflammation, gastrointestinal tract disorders, cell proliferation diseases and/or diabetes mellitus. See for example WO03/063800, WO03/042214, WO 03/035639, WO02/42298, EP 1283056, WO 01/16134, WO 01/02400, WO01/60350 or WO 00/73307.

It has now been found that certain pyrimidin-2-amine derivatives are novel potent and selective antagonists the A_{2B} adenosine receptor and can therefore be used in the treatment or prevention of these diseases.

Further objectives of the present invention are to provide a method for preparing said compounds; pharmaceutical compositions comprising an effective amount of said compounds; the use of the compounds in the manufacture of a medicament for the treatment of pathological conditions or diseases susceptible of being improved by antagonism of the A_{2B} adenosine receptor; and methods of treatment of pathological conditions or diseases susceptible to amelioration by antagonism of the A_{2B} adenosine receptor comprising the administration of the compounds of the invention to a subject in need of treatment.

Thus, the present invention is directed to new pyrimidin-2-amine derivatives derivatives of formula (I)

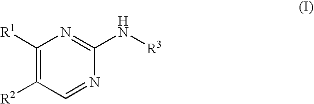

R¹ represents a monocyclic or polycyclic, aryl or heteroaryl group optionally substituted by one, two or three substituents selected from the group consisting of halogen atoms, straight or branched, optionally substituted lower alkyl, hydroxy, straight or branched, optionally substituted lower alkoxy, —SH, straight or branched optionally substituted lower alkylthio, nitro, cyano, —NR′R″, —CO₂R′, —C(O)—NR′R″, —N(R′″)C(O)—R′, —N(R′″)—C(O)NR′R″, wherein R′, R″ and R′″ each independently represents a hydrogen atom or a straight or branched, optionally substituted lower alkyl group or R′ and R″ together with the atom to which they are attached form a cyclic group;

R² represents a monocyclic N-containing heteroaryl group selected from the groups of formulae (IIa) or (IIb):

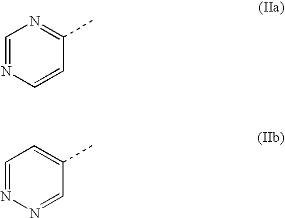

the groups of formula (IIa) and (IIb) being optionally substituted by one, two or three substituents selected from group consisting of halogen atoms, straight or branched, optionally substituted lower alkyl, hydroxy, straight or branched, optionally substituted lower alkoxy, —SH, straight or branched optionally substituted lower alkylthio, nitro, cyano, —NR′R″, —CO₂R′, —C(O)—NR′R″, —N(R′″)C(O)—R′, —N(R′″)—C(O)NR′R″, wherein R′, R″ and R′″ each independently represents a hydrogen atom or a straight or branched, optionally substituted lower alkyl group or R′ and R″ together with the atom to which they are attached form a cyclic group;

R³ represents a monocyclic or polycyclic, heteroaryl group being optionally substituted by one, two or three substituents selected from group consisting of halogen atoms, straight or branched, optionally substituted lower alkyl, hydroxy, oxo, straight or branched, optionally substituted lower alkoxy, —SH, straight or branched optionally substituted lower alkylthio, nitro, cyano, —NR′R″, —CO₂R′, —C(O)—NR′R″, —N(R′″)C(O)—R′, —N(R′″)—C(O)NR′R″, wherein R′, R″ and R′″ each independently represents a hydrogen atom or a straight or branched, optionally substituted lower alkyl group or R′ and R″ together with the atom to which they are attached form a cyclic group;

or an N-oxide or a pharmaceutically acceptable salt thereof.

As used herein the term lower alkyl embraces optionally substituted, linear or branched radicals having 1 to 8, preferably 1 to 6 and more preferably 1 to 4 carbon atoms. The substituents in said alkyl groups are selected from halogen atoms and hidroxy groups.

Examples include methyl, ethyl, n-propyl, i-propyl, n-butyl, sec-butyl and tert-butyl, n-pentyl, 1-methylbutyl, 2-methylbutyl, isopentyl, 1-ethylpropyl, 1,1-dimethylpropyl, 1,2-dimethylpropyl, n-hexyl, 1-ethylbutyl, 2-ethylbutyl, 1,1-dimethylbutyl, 1,2-dimethylbutyl, 1,3-dimethylbutyl, 2,2-dimethylbutyl, 2,3-dimethylbutyl, 2-methylpentyl, 3-methylpentyl and iso-hexyl radicals.

As used herein, the term lower alkoxy embraces optionally substituted, linear or brached oxy-containing radicals each having alkyl portions of 1 to 8, preferably 1 to 6 and more preferably 1 to 4 carbon atoms. The substituents in said alkoxy groups are selected from halogen atoms and hidroxy groups.

Preferred alkoxy radicals include methoxy, ethoxy, n-propoxy, i-propoxy, n-butoxy, sec-butoxy, t-butoxy, trifluoromethoxy, difluoromethoxy, hydroxymethoxy, 2-hydroxyethoxy or 2-hydroxypropoxy.

As used herein, the term lower alkylthio embraces radicals containing an optionally substituted, linear or brached alkyl radicals of 1 to 8, preferably 1 to 6 and more preferably 1 to 4 carbon atoms. The substituents in said alkylthio groups are selected from halogen atoms and hidroxy groups.

Preferred optionally substituted alkylthio radicals include methylthio, ethylthio, n-propylthio, i-propylthio, n-butylthio, sec-butylthio, t-butylthio, trifluoromethylthio, difluoromethylthio, hydroxymethylthio, 2-hydroxyethylthio or 2-hydroxypropylthio.

As used herein, the term cyclic group embraces, unless otherwise specified, carbocyclic and heterocyclic radicals. The cyclic radicals can contain one or more rings. Carbocyclic radicals may be aromatic or alicyclic, for example cycloalkyl radicals. Heterocyclic radicals also include heteroaryl radicals.

As used herein, the term aromatic group embraces typically a 5- to 14-membered aromatic ring system, such as a 5- or 6-membered ring which may contain one or more heteroatoms selected from O, S and N. When no heteroatoms are present the radical is named aryl radical and when at least one heteroatom is present it is named heteroaryl radical. The aromatic radical can be monocyclic or polycyclic, such as phenyl or naphthyl. When an aromatic radical or moiety carries 2 or more substituents, the substituents may be the same or different.

As used herein, the term aryl radical embraces typically a C₅-C₁₄ monocyclic or polycyclic aryl radical such as phenyl or naphthyl, anthranyl or phenanthryl. Phenyl is preferred. When an aryl radical carries 2 or more substituents, the substituents may be the same or different.

As used herein, the term heteroaryl radical embraces typically a 5- to 14-membered ring system comprising at least one heteroaromatic ring and containing at least one heteroatom selected from O, S and N. A heteroaryl radical may be a single ring or two or more fused rings wherein at least one ring contains a heteroatom.

Examples include pyridyl, pyrazinyl, pyrimidinyl, pyridazinyl, furyl, oxadiazolyl, oxazolyl, imidazolyl, thiazolyl, thiadiazolyl, thienyl, pyrrolyl, pyridinyl, benzothiazolyl, indolyl, indazolyl, purinyl, quinolyl, isoquinolyl, phthalazinyl, naphthyridinyl, quinoxalinyl, quinazolinyl, quinolizinyl, cinnolinyl, triazolyl, indolizinyl, indolinyl, isoindolinyl, isoindolyl, imidazolidinyl, pteridinyl and pyrazolyl radicals. Pyridyl, thienyl, furanyl, pyridazinyl, pyrimidinyl and quinolyl radicals are preferred.

When a heteroaryl radical carries 2 or more substituents, the substituents may be the same or different.

As used herein, some of the atoms, radicals, moieties, chains or cycles present in the general structures of the invention are “optionally substituted”. This means that these atoms, radicals, moieties, chains or cycles can be either unsubstituted or substituted in any position by one or more, for example 1, 2, 3 or 4, substituents, whereby the hydrogen atoms bound to the unsubstituted atoms, radicals, moieties, chains or cycles are replaced by chemically acceptable atoms, radicals, moieties, chains or cycles. When two or more substituents are present, each substituent may be the same or different.

As used herein, the term halogen atom embraces chlorine, fluorine, bromine or iodine atoms typically a fluorine, chlorine or bromine atom, most preferably chlorine or fluorine. The term halo when used as a prefix has the same meaning.

As used herein, the term pharmaceutically acceptable salt embraces salts with a pharmaceutically acceptable acid or base. Pharmaceutically acceptable acids include both inorganic acids, for example hydrochloric, sulphuric, phosphoric, diphosphoric, hydrobromic, hydroiodic and nitric acid and organic acids, for example citric, fumaric, maleic, malic, mandelic, ascorbic, oxalic, succinic, tartaric, benzoic, acetic, methanesulphonic, ethanesulphonic, benzenesulphonic or p-toluenesulphonic acid. Pharmaceutically acceptable bases include alkali metal (e.g. sodium or potassium) and alkali earth metal (e.g. calcium or magnesium) hydroxides and organic bases, for example alkyl amines, arylalkyl amines and heterocyclic amines.

Other preferred salts according to the invention are quaternary ammonium compounds wherein an equivalent of an anion (X—) is associated with the positive charge of the N atom. X— may be an anion of various mineral acids such as, for example, chloride, bromide, iodide, sulphate, nitrate, phosphate, or an anion of an organic acid such as, for example, acetate, maleate, fumarate, citrate, oxalate, succinate, tartrate, malate, mandelate, trifluoroacetate, methanesulphonate and p-toluenesulphonate. X— is preferably an anion selected from chloride, bromide, iodide, sulphate, nitrate, acetate, maleate, oxalate, succinate or trifluoroacetate. More preferably X— is chloride, bromide, trifluoroacetate or methanesulphonate.

As used herein, an N-oxide is formed from the tertiary basic amines or imines present in the molecule, using a convenient oxidising agent.

Preferred compounds of the invention are those wherein R² represents a pyrimidinyl or pyridazinyl group which may be optionally substituted by one, two or three substituents selected from group consisting of halogen atoms, straight or branched, optionally substituted lower alkyl, hydroxy, straight or branched, optionally substituted lower alkoxy, —SH, straight or branched optionally substituted lower alkylthio, nitro, cyano, —NR′R″, —CO₂R′, —C(O)—NR′R″, —N(R′″)C(O)—R′, —N(R′″)—C(O)NR′R″, wherein R′, R″ and R′″ each independently represents a hydrogen atom or a straight or branched, optionally substituted lower alkyl group or R′ and R″ together with the atom to which they are attached form a cyclic group.

Further preferred compounds are those wherein R² represents a pyrimidinyl or pyridazinyl group which may be optionally substituted by a straight or branched optionally substituted lower alkylthio group. More preferably R² represents a group selected from pyrimidin-4-yl, 2-methylthio-pyrimidin-4-yl and pyridazin-4-yl.

Also preferred are compounds wherein R³ represents a either a monocyclic or polycyclic heteroaryl group comprising a nitrogen-containing six-membered ring or a monocyclic five-membered heteroaryl group not containing nitrogen in the ring structure, the heteroaryl groups being optionally substituted by one, two or three substituents selected from the group consisting of halogen atoms, straight or branched, optionally substituted lower alkyl, hydroxy, oxo, straight or branched, optionally substituted lower alkoxy, —SH, straight or branched optionally substituted lower alkylthio, nitro, cyano, —NR′R″, —CO₂R′, —C(O)—NR′R″, —N(R′″)C(O)—R′, —N(R′″)—C(O)NR′R″, wherein R′, R″ and R′″ each independently represents a hydrogen atom or a straight or branched, optionally substituted lower alkyl group or R′ and R″ together with the atom to which they are attached form a cyclic group.

Further preferred compounds of the invention are those wherein R³ represents a monocyclic or polycyclic heteroaryl group comprising a nitrogen-containing six-membered ring, the heteroaryl groups being optionally substituted by one, two or three substituents selected from the group consisting of halogen atoms, straight or branched, optionally substituted lower alkyl, hydroxy, oxo, straight or branched, optionally substituted lower alkoxy, —SH, straight or branched optionally substituted lower alkylthio, nitro, cyano, —NR′R″, —CO₂R′, —C(O)—NR′R″, —N(R′″)C(O)—R′, —N(R′″)—C(O)NR′R″, wherein R′, R″ and R′″ each independently represents a hydrogen atom or a straight or branched, optionally substituted lower alkyl group or R′ and R″ together with the atom to which they are attached form a cyclic group.

It is even more preferred that in the compounds of the present invention R³ represents a rest selected from the group consisting of pyridine, pyrimidine, pyridazine, isoquinoline, quinoline, naphthyridine, pyridine-2(1H)-one, furan and thiophene all of them optionally substituted by one, two or three substituents selected from the group consisting of halogen atoms, straight or branched, optionally substituted lower alkyl, hydroxy, oxo, straight or branched, optionally substituted lower alkoxy, —SH, straight or branched optionally substituted lower alkylthio, nitro, cyano, —NR′R″, —CO₂R′, —C(O)—NR′R″, —N(R′″)C(O)—R′, —N(R′″)—C(O)NR′R″, wherein R′, R″ and R′″ each independently represents a hydrogen atom or a straight or branched, optionally substituted lower alkyl group or R′ and R″ together with the atom to which they are attached form a cyclic group.

In a still more preferred execution R³ is selected from the group consisting of pyridine, pyrimidine, pyridazine, isoquinoline, quinoline, naphthyridine and pyridine-2(1H)-one, all of them optionally substituted by a substituent selected from the group consisting of halogen atoms, straight or branched, optionally substituted lower alkyl, oxo, straight or branched, optionally substituted lower alkoxy, straight or branched optionally substituted lower alkylthio and cyano groups.

More preferably R³ represents a group selected from the group consisting of pyridine and pyridine-2(1H)-one, all of them optionally substituted by a substituent selected from the group consisting of halogen atoms, straight or branched, optionally substituted lower alkyl, oxo, straight or branched, optionally substituted lower alkoxy, straight or branched optionally substituted lower alkylthio and cyano groups.

Still more preferably R³ represents a group selected from 1-oxidopyridin-3-yl, pyrimidin-5-yl, 5-methoxypyridin-3-yl, 6-methylpyridin-3-yl, pyrazin-2-yl, 5-cyano-pyridin-3-yl, 1-oxidopyrimidin-5-yl, 2-(methylthio)pyrimidin-4-yl, 6-(benzyloxy)pyridin-3-yl, 6-oxo-1,6-dihydropyridin-3-yl, 1,6-naphthyridin-8-yl, isoquinolin-4-yl, quinolin-3-yl, pyridin-3-yl, 6-methoxypyridin-3-yl, pyridin-2-yl, 6-fluoropyridin-3-yl, 4-methylpyridin-3-yl and pyridazin-4-yl. Still more preferably R³ represents a group selected from pyridin-3-yl, 6-methoxypyridin-3-yl, pyridin-2-yl, 6-fluoropyridin-3-yl, 4-methylpyridin-3-yl and pyridazin-4-yl.

Most preferably, R¹ represents a group selected from phenyl, furan-2-yl, furan-3-yl, thien-2-yl, thien-3-yl, pyridin-2-yl, pyridin-3-yl and pyridin-4-yl all of them optionally substituted by one, two or three substituents selected from the group consisting of halogen atoms, straight or branched, optionally substituted lower alkyl, hydroxy, straight or branched, optionally substituted lower alkoxy, —SH, straight or branched optionally substituted lower alkylthio, nitro, cyano, —NR′R″, —CO₂R′, —C(O)—NR′R″, —N(R′″)C(O)—R′, —N(R′″)—C(O)NR′R″, wherein R′, R″ and R′″ each independently represents a hydrogen atom or a straight or branched, optionally substituted lower alkyl group or R′ and R″ together with the atom to which they are attached form a cyclic group. More preferably R¹ represents a group selected from phenyl, furan-2-yl, furan-3-yl, thien-2-yl, all of them optionally substituted by an halogen atom. Still more preferably R¹ represents a group selected from furan-2-yl, thien-2-yl and 3-fluorophenyl and most preferably selected from unsubstituted furan-2-yl and unsubstituted thien-2-yl.

Preferred compounds of the present invention are those wherein R² represents a pyrimidinyl or pyridazinyl group which may be optionally substituted by one, two or three substituents selected from group consisting of halogen atoms, straight or branched, optionally substituted lower alkyl, hydroxy, straight or branched, optionally substituted lower alkoxy, —SH, straight or branched optionally substituted lower alkylthio, nitro, cyano, —NR′R″, —CO₂R′, —C(O)—NR′R″, —N(R′″)C(O)—R′, —N(R′″)—C(O)NR′R″, wherein R′, R″ and R′″ each independently represents a hydrogen atom or a straight or branched, optionally substituted lower alkyl group or R′ and R″ together with the atom to which they are attached form a cyclic group.

Typically, R² represents a monocyclic heteroaryl group of formula (IIa): 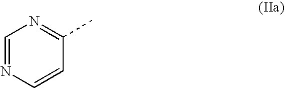
the heteroaryl group of formula (IIa) being optionally substituted by one, two or three substituents selected from group the group consisting of halogen atoms, straight or branched, optionally substituted lower alkyl, hydroxy, oxo, straight or branched, optionally substituted lower alkoxy, —SH, straight or branched optionally substituted lower alkylthio, nitro, cyano, —NR′R″, —CO₂R′, —C(O)—NR′R″, —N(R′″)C(O)—R′, —N(R′″)—C(O)NR′R″, wherein R′, R″ and R′″ each independently represents a hydrogen atom or a straight or branched, optionally substituted lower alkyl group or R′ and R″ together with the atom to which they are attached form a cyclic group and wherein R³ represents a pyridine group optionally substituted by one, two or three substituents selected from group consisting of halogen atoms, straight or branched, optionally substituted lower alkyl, hydroxy, oxo, straight or branched, optionally substituted lower alkoxy, —SH, straight or branched optionally substituted lower alkylthio, nitro, cyano, —NR′R″, —CO₂R′, —C(O)—NR′R″, —N(R′″)C(O)—R′, —N(R′″)—C(O)NR′R″, wherein R′, R″ and R′″ each independently represents a hydrogen atom or a straight or branched, optionally substituted lower alkyl group or R′ and R″ together with the atom to which they are attached form a cyclic group.

Further preferred compounds of the present invention are those wherein R²represents a pyrimidinyl or pyridazinyl group which may be optionally substituted by a straight or branched optionally substituted lower alkylthio group. Most preferred are the compound wherein R² represents an unsubstituted pyrimidin-4-yl or unsubstituted pyridazin-4-yl group.

Particularly preferred compounds of the present invention are those wherein R¹ represents a group selected from phenyl, furan-2-yl, furan-3-yl and thien-2-yl, all of them optionally substituted by an halogen atom, R² represents a pyrimidinyl or pyridazinyl group which may be optionally substituted by a straight or branched optionally substituted lower alkylthio group and wherein R³ is selected from the group consisting of pyridine, pyrimidine, pyridazine, isoquinoline, quinoline, naphthyridine and pyridine-2(1H)-one, all of them optionally substituted by a substituent selected from the group consisting of halogen atoms, straight or branched, optionally substituted lower alkyl, oxo, straight or branched, optionally substituted lower alkoxy, straight or branched optionally substituted lower alkylthio and cyano groups as well as their pharmaceutically acceptable salts and N-oxides.

Still more preferred are the compounds wherein R¹ represents a group selected from unsubstituted furan-2-yl and unsubstituted thien-2-yl, R² represents an unsubstituted pyrimidin-4-yl or an unsubstituted pyridazin-4-yl and wherein R³ is selected from the group consisting of pyridine, pyrimidine, pyridazine, isoquinoline, quinoline, naphthyridine and pyridine-2(1H)-one, all of them optionally substituted by a substituent selected from the group consisting of halogen atoms, straight or branched, optionally substituted lower alkyl, oxo, straight or branched, optionally substituted lower alkoxy, straight or branched optionally substituted lower alkylthio and cyano groups as well as their pharmaceutically acceptable salts and N-oxides.

Particular individual compounds of the invention include:

4′-(2-furyl)-N-pyridin-3-yl-4,5′-bipyrimidin-2′-amine

4′-(2-furyl)-N-(6-methoxypyridin-3-yl)-4,5′-bipyrimidin-2′-amine

4′-(2-furyl)-N-pyridin-2-yl-4,5′-bipyrimidin-2′-amine

N-(6-fluoropyridin-3-yl)-4′-(2-furyl)-4,5′-bipyrimidin-2′-amine

4′-(2-furyl)-N-(4-methylpyridin-3-yl)-4,5′-bipyrimidin-2′-amine

N-pyridin-3-yl-4′-thien-2-yl-4,5′-bipyrimidin-2′-amine

4′-(3-fluorophenyl)-N-pyridin-3-yl-4,5′-bipyrimidin-2′-amine

4′-(3-fluorophenyl)-N-(6-methoxypyridin-3-yl)-4,5′-bipyrimidin-2′-amine

4′-(2-furyl)-N-(6-methoxypyridin-3-yl)-2-(methylthio)-4,5′-bipyrimidin-2′-amine

4′-(3-fluorophenyl)-2-(methylthio)-N-pyridin-3-yl-4,5′-bipyrimidin-2′-amine

4-(2-furyl)-5-pyridazin-4-yl-N-pyridin-3-ylpyrimidin-2-amine

4′-(2-furyl)-N-(1-oxidopyridin-3-yl)-4,5′-bipyrimidin-2′-amine

4′-(2-furyl)-N-pyrimidin-5-yl-4,5′-bipyrimidin-2′-amine

4′-(2-furyl)-N-(5-methoxypyridin-3-yl)-4,5′-bipyrimidin-2′-amine

4′-(2-furyl)-N-(6-methylpyridin-3-yl)-4,5′-bipyrimidin-2′-amine

4′-(2-furyl)-N-pyrazin-2-yl-4,5′-bipyrimidin-2′-amine

5-{[4′-(2-furyl)-4,5′-bipyrimidin-2′-yl]amino}nicotinonitrile

4′-(2-furyl)-N-(1-oxidopyrimidin-5-yl)-4,5′-bipyrimidin-2′-amine

4′-(2-furyl)-N-[2-(methylthio)pyrimidin-4-yl]-4,5′-bipyrimidin-2′-amine

N-[6-(benzyloxy)pyridin-3-yl]-4′-(2-furyl)-4,5′-bipyrimidin-2′-amine

5-{[4′-(2-furyl)-4,5′-bipyrimidin-2′-yl]amino}pyridin-2(1H)-one

4′-(2-furyl)-N-1,6-naphthyridin-8-yl-4,5′-bipyrimidin-2′-amine

4′-(2-furyl)-N-isoquinolin-4-yl-4,5′-bipyrimidin-2′-amine

4′-(2-furyl)-N-quinolin-3-yl-4,5′-bipyrimidin-2′-amine

4′-(3-furyl)-N-pyridin-3-yl-4,5′-bipyrimidin-2′-amine

4′-(3-furyl)-N-pyrimidin-5-yl-4,5′-bipyrimidin-2′-amine

N-pyrimidin-5-yl-4′-(2-thienyl)-4,5′-bipyrimidin-2′-amine

N-(1-oxidopyridin-3-yl)-4′-(2-thienyl)-4,5′-bipyrimidin-2′-amine

5-pyridazin-4-yl-N-pyridin-3-yl-4-(2-thienyl)pyrimidin-2-amine

4-(2-furyl)-5-pyridazin-4-yl-N-pyrimidin-5-ylpyrimidin-2-amine

Of outstanding interest are:

4′-(2-furyl)-N-pyridin-3-yl-4,5′-bipyrimidin-2′-amine

4′-(2-furyl)-N-(6-methoxypyridin-3-yl)-4,5′-bipyrimidin-2′-amine

N-(6-fluoropyridin-3-yl)-4′-(2-furyl)-4,5′-bipyrimidin-2′-amine

N-pyridin-3-yl-4′-thien-2-yl-4,5′-bipyrimidin-2′-amine

4-(2-furyl)-5-pyridazin-4-yl-N-pyridin-3-ylpyrimidin-2-amine

4′-(2-furyl)-N-(1-oxidopyridin-3-yl)-4,5′-bipyrimidin-2′-amine

4′-(2-furyl)-N-pyrimidin-5-yl-4,5′-bipyrimidin-2′-amine

5-{[4′-(2-furyl)-4,5′-bipyrimidin-2′-yl]amino}pyridin-2(1H)-one

According to a further feature of the present invention, compounds of general formula (I) are prepared by coupling a compound of formula (IX) where R¹ and R² are as hereinbefore defined with a compound of formula (III) where R³ is as hereinbefore defined and X is halogen, preferably bromine, iodine or chlorine.

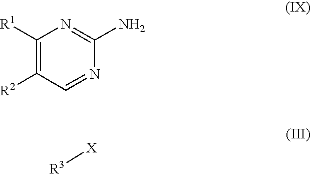

The reaction is carried out using the palladium and/or copper catalyzed general methods for the arylation of amines (for references see Yin, J. et al. Org. Lett. 2002, 4(20), 3481 and Buchwald S. L. et al. J. Am. Chem. Soc. 2002, 124, 7421).

The intermediate compounds of formula (IX) can be prepared by reaction of a corresponding ethanone derivative (IV) in a two steps sequence.

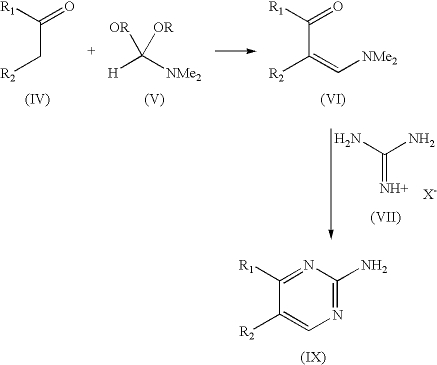

First the compound of formula (IV) is reacted in neat dimethylformamide dialkyl acetal of formula (V) (preferably dimethylacetal) at room temperature. Then the corresponding dimethylamino propenone derivative of formula (VI) reacts with guanidine in the form of a salt (VII), e.g. hydrohalide or carbonate, in an organic solvent, preferably a polar aprotic solvent, such as N,N-dimethylformamide, dioxane, acetone or tetrahydrofuran, in the presence of a base, such as potassium carbonate, and at a temperature from 15° C. to 110° C. to yield the compound of formula (IX).

The intermediate compounds of formula (IV) can be prepared by reaction of a methyl substituted heteroaromatic ring (X) with and aromatic or heteroaromatic carboxylic acid ester (preferably methyl or ethyl ester) (XI) as shown in the scheme below.

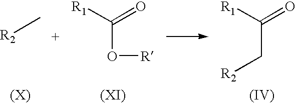

The reaction is carried out in an organic solvent, preferably a polar aprotic solvent such as tetrahydrofuran, in the presence of a base such as lithium bis(trimethylsilyl)amide and at a temperature from −70° C. to 50° C. to yield the compound of formula (IV).

Alternatively, compounds of general formula (I) can be prepared by condensation of the corresponding dimethylamino propenone derivative of formula (VI) with substituted guanidines of general formula (VIII) following the scheme:

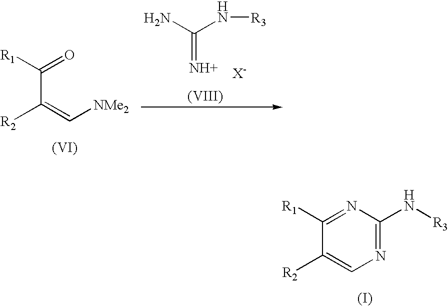

Guanidines of general formula (VIII) are prepared using methods known per se (for example see Barber, C. G. et al. Bioorg. Med. Chem. Lett. 2002, 12, 181-184).

When the groups R¹ to R³ are susceptible to chemical reaction under the conditions of the hereinbefore described processes or are incompatible with said processes, alternative processes can be readily carried out utilising organic synthetic chemistry methods to, for example, protect functional groups and finally eliminate protecting groups.

The pyrimidin-2-amine derivatives of formula (I) can be converted by methods known per se into pharmaceutically acceptable salts or N-oxides. Preferred salts are acid addition salts obtainable by treatment with organic or inorganic acids such as fumaric, tartaric, succinic or hydrochloric acid. Also pyrimidin-2-amine derivatives of formula (I) in which there is the presence of an acidic group may be converted into pharmacologically acceptable salts by reaction with an alkali metal hydroxide or an organic base such as sodium or potassium hydroxide. The acid or alkali addition salts so formed may be interchanged with suitable pharmaceutically acceptable counter ions using processes known per se.

Adenosine I Receptor Subtype Competition Radioligand Binding Assay

CHO-K1 cells expressing human recombinant A₁ receptors were purchased from Euroscreen (Belgium). For membrane preparation, cells were collected from tissue culture plates using a cell scraper, resuspended in 10-15 ml of homogenization buffer (Tris-HCl 15 mM pH 7.5, MgCl₂ 2 mM, EDTA 0.3 mM, EGTA 1 mM), homogenized, and centrifuged at 40.000 g for 25 minutes. The resulting pellet was resuspended in the same buffer and centrifuged again for 25 minutes. Finally, the pellet was resuspended in 500 μl of storage buffer (Tris-HCl 7.5 mM pH 7.5, MgCl₂ 12.5 mM, EDTA 0.3 mM, EGTA 1 mM, sucrose 250 mM) where the total protein content was determined.

Competition assays were carried out incubating 15 μg of A₁-membrane preparations, 2 nM [³H]-DPCPX (Amersham) as radioligand and 10 μM of unlabeled DPCPX ligand, in a total volume of 100 μl of buffer (Hepes 20 mM pH 7.4, NaCl 100 mM, MgCl₂ 10 mM, 2 U/ml adenosin deaminase) for 1 h at 25° C. Samples were filtered and washed 4 times with 250 μl of buffer (Hepes 20 mM pH 7.4, NaCl 100 mM, MgCl₂ 10 mM) using plates (Millipore MAFCN0B50) preincubated for 15 min. in 250 μl of the same buffer. Samples were counted using 30 μl of LSC Universol (ICN) in a Wallac 1450 Microbeta counter. Non specific binding was tested using 10 μM R-PIA.

Adenosine 2A Receptor Subtype Competition Radioligand Binding Assay

Membranes were prepared from Hela cells stably transfected with the human recombinant A_{2A} receptor. For membrane preparation, cells were collected from tissue culture plates using a cell scraper, resuspended in 10-15 ml homogenization buffer (Tris-HCl 5 mM, EDTA 2 mM), homogenized, and centrifuged at 1.000 g. for 10 min. at 4° C. The supernatant was then recovered and centrifuged at 50.000 g for 1 h. at 4° C. Finally, the pellet was resuspended in 100-500 μl storage buffer (Tris-HCl 50 mM pH 7.4) where the total protein content was determined.

Competition assays were carried out incubating 5 μg of A_{2A}-membranes, 3 nM [³H]-ZM241385 (Tocris) as radioligand and 50 μM of unlabeled ZM241385 ligand, in a total volume of 100 μl of buffer (TrisHCl 50 μM pH 7.4, EDTA 1 mM, MgCl₂ 10 mM, 2 U/ml adenosin deaminase) for 30 minutes at 25° C. Samples were then filtered and washed 4 times with 250 μl of buffer (TrisHCl 50 μM pH 7.4, EDTA 1 mM, MgCl₂ 10 mM) using plates (Millipore MAFCN0B50) preincubated for 15 min. in 250 μl of the same buffer. Samples were counted using 30 μl of LSC Universol (ICN) in a Wallac 1450 Microbeta counter. Non specific binding was tested using 50 μM NECA.

Adenosine 2B Receptor Subtype Competition Radioligand Binding Assay

Membranes derived from HEK293 cells transfected with recombinant human A_{2B} were purchased from Receptor Biology. Competition assays were carried out incubating 18 μg of A_{2B}-membranes, 35 nM [³H]-DPCPX (Amersham) as radioligand and 400 μM of unlabeled DPCPX ligand, in a total volume of 100 μl of buffer (Tris-HCl 50 mM pH 6.5, MgCl₂ 10 mM, EDTA 1 mM, benzamidine 0.1 mM, 2 U/ml adenosine deaminase) for 30 minutes at 25° C. Samples were filtered 4 times with 250 μl of buffer (Tris-HCl 50 mM pH 6.5) using plates (Millipore MAFBN0B50) preincubated for 15 min. in 250 μl of the same buffer. Samples were counted using 30 μl of LSC Universol (ICN) in a Wallac 1450 Microbeta counter. Non specific binding was tested using 400 μM NECA.

Adenosine 3 Receptor Subtype Competition Radioligand Binding Assay

Membranes were prepared from Hela cells stably transfected with the human recombinant A₃ receptor. For membrane preparation, cells were collected from tissue culture plates using a cell scraper, resuspended in 10-15 ml of homogenization buffer (Tris-HCl 5 mM, EDTA 2 mM), homogenized, and centrifuged at 1.000 g. for 10 min. at 4° C. The supernatant was then recovered and centrifuged at 50.000 g for 1 h. at 4° C. Finally, the pellet was resuspended in 100-500 μl of storage buffer (Tris-HCl 50 mM pH 7.4) where the total protein content was determined.

Competition assays were carried out incubating 100 μg of A₃-membranes, 30 nM [³H]-NECA (Amersham) as radioligand and 50 μM of unlabeled NECA ligand, in a total volume of 100 μl of buffer (Tris-HCl 50 mM pH 7.4, MgCl₂ 5 mM, EDTA 1 mM, 2 U/ml adenosine deaminase) for 3 hours at 25° C. Samples were filtered 4 times with 250 μl of buffer (Tris-HCl 50 mM pH 7.4) using plates (Millipore MAFBN0B50) preincubated for 15 min. in 250 μl of the same buffer. Samples were counted using 30 μl of LSC Universol (ICN) in a Wallac 1450 Microbeta counter. Non specific binding was tested using 100 μM of R-PIA.

Adenosine 2B Receptor Subtype Functional Cellular cAMP Assay

The assay was carried out using CHO-K1 transfected with human recombinant A_{2B} receptor and a commercial EIA kit (Amersham, RPN225). Cells were seeded in 96 well plates at 10.000 cells/well. After 24 h, plates were placed on ice for 5 minutes, the medium was removed, and all wells were rinsed twice with 100 μl of incubation medium (Hepes 25 mM, DMEM-F12). After washing, Rolipram (30 μM) and antagonists were added in 100 μl of incubation medium, and the plates were incubated for 15 minutes at 37° C. NECA was then added to reach a final concentration of 10 μM and the plates were incubated for another 15 minutes at 37° C. After incubation, medium was removed from all wells, 200 μl of lysis buffer (reactive 1B from Amersham RPN225) were added, and the plates were incubated 10 minutes at room temperature with slight agitation. After lysis, 100 μl of the lysate were transferred to a plate pretreated with anti-rabbit antibody, 100 μl of rabbit anti-cAMP serum were added to the wells and the plates were incubated for 2 h at 4° C. Peroxidase-coupled cAMP was then added, and the plates incubated for 1 hour at 4° C. Plates were then washed 4 times with 100 μl of buffer (washing buffer, Amersham RPN225). After washing, 150 μl of peroxidase substrate were added to the wells and the plates were incubated for 1 hour at room temperature. Finally, 100 μl of 1 M sulfuric acid were added to stop the reaction and the OD was measured at 450-495 nM.

The results are shown in Table 1. Functional Kᵢ was calculated using the following formula (Cheng Y. C. And Prusoff W. H. Biochem. Pharmacol. 1973, 22, 3099-3108): Kᵢ (cAMP, nM)=[IC₅₀/(1+([C]/K_{d}))], where IC₅₀ is the IC₅₀ for the test compound, [C] is the total NECA concentration and K_{d} is the EC₅₀ for NECA.

TABLE 1

| | Binding | Binding | cAMP assay | Binding |
|---|---|---|---|---|
| | human A₁ | human A_{2A} | human A_{2B} | human A₃ |
| Example | Kᵢ (nM) | Kᵢ (nM) | Kᵢ* (nM) | Kᵢ (nM) |
| 1 | 14% @ 1 μM | 2537 | 17 | 1096 |
| 2 | 5% @ 1 μM | 14% @ 1 μM | 55 | 4315 |
| 4 | 30% @ 1 μM | 21% @ 1 μM | 100 | 1692 |
| 6 | 31% @ 1 μM | 620 | 12 | 310 |
| 11 | 23% @ 1 μM | 17% @ 1 μM | 70 | 407 |
| 12 | 4% @ 1 μM | 7% @ 1 μM | 57 | 846 |
| 13 | 3% @ 1 μM | 15% @ 1 μM | 12 | 1442 |
| 21 | 0% @ 1 μM | 0% @ 1 μM | 17 | 2787 |

*Functional Kᵢ

It can be seen from Table 1 that the compounds of formula (I) are potent inhibitors of the A_{2B} adenosine receptor subtype and very selective over the other adenosine receptor subtypes. Preferred pyrimidin-2-amine derivatives of the invention possess a functional Kᵢ value for the inhibition of A_{2B} (determined as defined above) of less than 100 nM, preferably less than 60 nM and most preferably less than 20 nM.

The pyrimidin-2-amine derivatives of the invention are useful in the treatment or prevention of diseases known to be susceptible to improvement by treatment with an antagonist of the A_{2B} adenosine receptor. Such diseases are, for example asthma, bronchoconstriction, allergic diseases, inflammation, reperfusion injury, myocardial ischemia, atherosclerosis, hypertension, retinopathy, diabetes mellitus, inflammation, gastrointestinal tract disorders, and/or autoimmune diseases. Examples of autoimmune diseases which can be treated or prevented using the compounds of the invention are Addison's disease, autoimmune hemolytic anemia, Crohn's disease, Goodpasture's syndrome, Graves disease, Hashimoto's thyroiditis, idiopathic thrombocytopenic purpura, insulin-dependent diabetes mellitus, multiple sclerosis, myasthenia gravis, pemphigus vulgaris, pernicious anemia, poststreptococcal glomerulonephritis, psoriasis, rheumatoid arthritis, scleroderma, Sjogren's syndrome, spontaneous infertility, and systemic lupus erythematosus.

Accordingly, the pyrimidin-2-amine derivatives of the invention and pharmaceutically acceptable salts thereof, and pharmaceutical compositions comprising such compound and/or salts thereof, may be used in a method of treatment of disorders of the human body which comprises administering to a subject requiring such treatment an effective amount of pyrimidin-2-amine derivative of the invention or a pharmaceutically acceptable salt thereof.

The present invention also provides pharmaceutical compositions which comprise, as an active ingredient, at least a pyrimidin-2-amine derivative of formula (I) or a pharmaceutically acceptable salt thereof in association with a pharmaceutically acceptable excipient such as a carrier or diluent. The active ingredient may comprise 0.001% to 99% by weight, preferably 0.01% to 90% by weight of the composition depending upon the nature of the formulation and whether further dilution is to be made prior to application. Preferably the compositions are made up in a form suitable for oral, topical, nasal, rectal, percutaneous or injectable administration.

The pharmaceutically acceptable excipients which are admixed with the active compound, or salts of such compound, to form the compositions of this invention are well-known per se and the actual excipients used depend inter alia on the intended method of administering the compositions.

Compositions of this invention are preferably adapted for injectable and per os administration. In this case, the compositions for oral administration may take the form of tablets, retard tablets, sublingual tablets, capsules, inhalation aerosols, inhalation solutions, dry powder inhalation, or liquid preparations, such as mixtures, elixirs, syrups or suspensions, all containing the compound of the invention; such preparations may be made by methods well-known in the art.

The diluents which may be used in the preparation of the compositions include those liquid and solid diluents which are compatible with the active ingredient, together with colouring or flavouring agents, if desired. Tablets or capsules may conveniently contain between 2 and 500 mg of active ingredient or the equivalent amount of a salt thereof.

The liquid composition adapted for oral use may be in the form of solutions or suspensions. The solutions may be aqueous solutions of a soluble salt or other derivative of the active compound in association with, for example, sucrose to form a syrup. The suspensions may comprise an insoluble active compound of the invention or a pharmaceutically acceptable salt thereof in association with water, together with a suspending agent or flavouring agent.

Compositions for parenteral injection may be prepared from soluble salts, which may or may not be freeze-dried and which may be dissolved in pyrogen free aqueous media or other appropriate parenteral injection fluid.

Effective doses are normally in the range of 2-2000 mg of active ingredient per day. Daily dosage may be administered in one or more treatments, preferably from 1 to 4 treatments, per day.

The syntheses of the compounds of the invention and of the intermediates for use therein are illustrated by the following Examples (1 to 11) including Preparation Examples (Preparations 1-6) which do not limit the scope of the invention in any way.

¹H Nuclear Magnetic Resonance Spectra were recorded on a Varian Gemini 300 spectrometer. Melting points were recorded using a Büchi B-540 apparatus. The chromatographic separations were obtained using a Waters 2795 system equipped with a Symmetry C18 (2.1×100 mm, 3.5 mm) column. As detectors a Micromass ZMD mass spectrometer using ES ionization and a Waters 996 Diode Array detector were used. The mobile phase was formic acid (0.46 ml), ammonia (0.115 ml) and water (1000 ml) (A) and formic acid (0.4 ml), ammonia (0.1 ml), methanol (500 ml) and acetonitrile (500 ml) (B): initially from 0% to 95% of B in 20 min, and then 4 min. with 95% of B. The reequilibration time between two injections was 5 min. The flow rate was 0.4 ml/min. The injection volume was 5 μl. Diode array chromatograms were processed at 210 nm.

### PREPARATION EXAMPLES

Preparation 1

### 4′-(2-Furyl)-4,5′-bipyrimidin-2′-amine

A mixture of 3-(dimethylamino)-1-(2-furyl)-2-pyrimidin-4-ylprop-2-en-1-one (1.54 g, 6.33 mmol), K₂CO₃ (5.24 g, 38 mmol) and guanidine hydrochloride (1.81 g, 19 mmol) in DMF (12 mL) was heated to 70° C. for 20 hours and then allowed to cool to room temperature. Water was added, the precipitate was collected by filtration and washed copiously with water. The solid is dried under vacuum to yield the title compound (920 mg, 61%).

m.p.: 221.5-221.8° C.

δ ¹H-NMR (DMSO-d₆): 9.17 (s, 1H), 8.74 (d, 1H), 8.46 (s, 1H), 7.67 (s, 1H), 7.36 (dd, 1H), 7.18 (s, 2H), 6.92 (d, 1H), 6.61 (dd, 1H).

ESI/MS m/e: 240 ([M+H]⁺, C₁₂H₉N₅O).

Retention time (min.): 7

### 3-(Dimethylamino)-1-(2-furyl)-2-pyrimidin-4-ylprop-2-en-1-one

A suspension of 1-(2-furyl)-2-pyrimidin-4-ylethanone (1.59 g, 8.45 mmol) in N,N-dimethylformamide dimethyl acetal (4.5 mL, 33.8 mmol) was heated to 100° C. for 2 hours. The mixture was allowed to cool to room temperature, the solvent was evaporated under reduced pressure and the residue was partitioned between ethyl acetate and a saturated solution of ammonium chloride. The aqueous phase was extracted with ethyl acetate, the organic extracts were washed with brine, dried (Na₂SO₄) and evaporated under reduced pressure to provide the title compound as a red oil (1.54g, 75%).

δ ¹H-NMR (CDCl₃): 9.01 (s, 1H), 8.38 (d, 1H), 7.81 (s, 1H), 7.43 (s, 1H), 7.05 (d, 1H), 6.90 (d, 1H), 6.43 (d, 1H), 2.98 (s, 6H).

ESI/MS m/e: 244 ([M+H]⁺, C₁₃H₁₃N₃O₂)

### 1-(2-Furyl)-2-pyrimidin-4-ylethanone

To a solution of 4-methylpyrimidine (0.93 g, 9.9 mmol) and ethyl 2-furoate (1.54 g, 11 mmol) in anhydrous THF (8 mL) at 0° C., under Ar, was added dropwise via syringe pump (1 hour) a solution of lithium bis(trimethylsilyl)amide (1M solution in hexanes, 20 mL). The resulting mixture was stirred at room temperature for 2 hours. The precipitate was collected by filtration, washed with a saturated aqueous solution of ammonium chloride and water, then dried under vacuum to yield the title compound as a yellow solid (1.59 g, 85%).

ESI/MS m/e: 189 ([M+H]⁺, C₁₀H₈N₂O₂)

Preparation 2

### 4′-Thien-2-yl-4,5′-bipyrimidin-2′-amine

Obtained as a brownish solid (80% overall) from 4-methylpyrimidine and ethyl 2-thiophenecarboxylate following the procedure described in Preparation 1.

m.p.: 207-208° C.

δ ¹H-NMR (DMSO-d₆): 9.22 (s, 1H), 8.77 (d, 1H), 8.37 (s, 1H), 7.70 (m, 1H), 7.53 (dd, 1H), 7.15 (s, 2H), 6.97 (m, 1H), 6.80 (m, 1H).

ESI/MS m/e: 256 ([M+H]⁺, C₁₂H₉N₅S).

Retention time (min.): 9

Preparation 3

### 4′-(3-Fluorophenyl)-4,5′-bipyrimidin-2′-amine

Obtained as a brownish solid (45% overall) from 4-methylpyrimidine and ethyl 3-fluorobenzoate following the procedure described in Preparation 1.

m.p.: 202.6-203.9° C.

δ ¹H-NMR (DMSO-d₆): 9.09 (s, 1H), 8.64 (d, 1H), 8.59 (s, 1H), 7.37 (m, 1H), 7.31 (s, 2H), 7.26 (m, 1H), 7.19 (m, 1H), 7.14 (dd, 1H), 7.06 (m, 1H).

ESI/MS m/e: 268 ([M+H]⁺, C₁₄H₁₀FN₅).

Retention time (min.): 9

Preparation 4

### 4′-(2-Furyl)-2-(methylthio)-4,5′-bipyrimidin-2′-amine

Obtained as a beige solid (51% overall) from 4-methyl-2-(methylthio)pyrimidine and ethyl 2-furoate following the procedure described in Preparation 1.

ESI/MS m/e: 286 ([M+H]⁺, C₁₃H₁₁N₅OS).

Retention time (min.): 6.8

Preparation 5

### 4′-(3-Fluorophenyl)-2-(methylthio)-4,5′-bipyrimidin-2′-amine

Obtained as an orange solid (30% overall) from 4-methyl-2-(methylthio)pyrimidine and ethyl 3-fluorobenzoate following the procedure described in Preparation 1.

m.p.: 158.7-159.7° C.

δ ¹H-NMR (DMSO-d₆): 8.67 (s, 1H), 8.44 (d, 1H), 7.39 (m, 1H), 7.35 (s, 2H), 7.27 (m, 1H), 7.20 (m, 1H), 7.08 (d, 1H), 6.97 (d, 1H), 3.34 (s, 3H).

ESI/MS m/e: 314 ([M+H]⁺, C₁₅H₁₂FN₅S).

Retention time (min.): 7

Preparation 6

### 4-(2-Furyl)-5-pyridazin-4-ylpyrimidin-2-amine

Obtained as an orange solid (10% overall) from 4-methylpyridazine and ethyl 2-furoate following the procedure described in Preparation 1.

m.p.: 195-196° C.

δ ¹H-NMR (DMSO-d₆): 9.22 (d, 1H), 9.08 (s, 1H), 8.32 (s, 1H), 7.67 (s, 1H), 7.65 (m, 1H), 7.13 (s, 2H), 6.90 (d, 1H), 6.60 (m, 1H).

ESI/MS m/e: 240 ([M+H]⁺, C₁₂H₉N₅O).

Retention time (min.): 6.2

Preparation 7

### 4′-(3-furyl)-4,5′-bipyrimidin-2′-amine

Obtained as a white solid (55% overall) from 4-methylpyrimidine and ethyl 3-furoate following the procedure described in Preparation 1.

δ ¹H-NMR (DMSO-d₆): 9.19 (d, 1H), 8.73 (d, 1H), 8.43 (s, 1H), 7.78 (s, 1H), 7.66 (t, 1H), 7.46 (dd, 1H), 7.09 (s, 2H), 6.34 (s, 1H).

ESI/MS m/e: 240 ([M+H]⁺, C₁₂H₉N₅O)

Retention time (min.): 7

Preparation 8

### 5-pyridazin-4-yl-4-(2-thienyl)pyrimidin-2-amine

Obtained as a yellow solid (30% overall) from 4-methylpyridazine and ethyl 2-thiophenecarboxylate following the procedure described in Preparation 1.

ESI/MS m/e: 256 ([M+H]⁺, C₁₂H₉N₅S)

Retention time (min.): 8

### EXAMPLES

TABLE 2

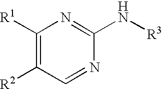

| Example | R¹ | R² | R³ |
|---|---|---|---|
| 1 | 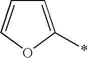 | 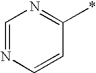 | 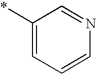 |
| 2 | 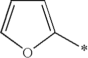 | 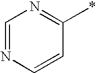 | 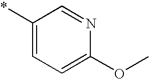 |
| 3 | 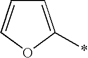 | 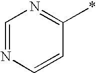 | 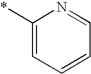 |
| 4 | 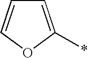 | 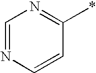 | 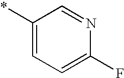 |
| 5 | 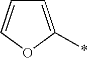 | 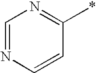 | 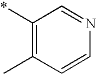 |
| 6 | 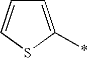 | 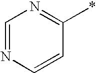 | 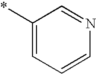 |
| 7 | 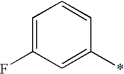 | 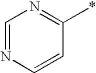 | 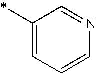 |
| 8 | 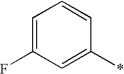 | 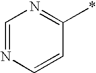 | 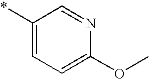 |
| 9 | 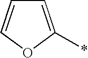 | 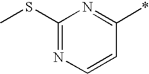 | 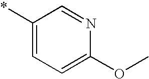 |
| 10 | 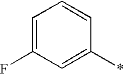 | 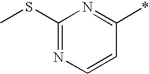 | 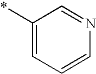 |
| 11 | 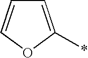 | 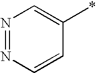 | 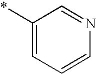 |
| 12 | 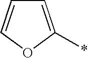 | 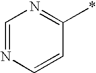 | 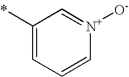 |
| 13 | 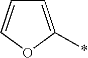 | 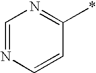 | 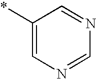 |
| 14 | 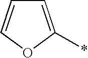 | 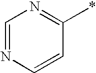 | 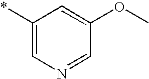 |
| 15 | 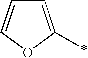 | 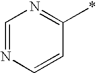 | 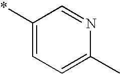 |
| 16 | 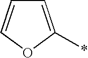 | 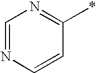 | 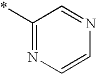 |
| 17 | 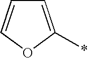 | 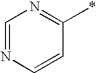 | 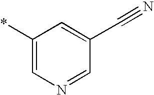 |
| 18 | 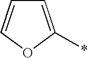 | 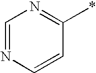 | 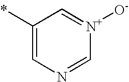 |
| 19 | 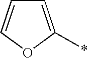 | 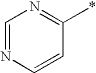 | 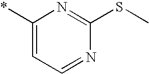 |
| 20 | 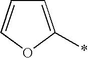 | 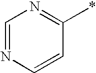 | 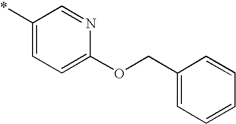 |
| 21 | 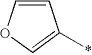 | 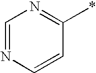 | 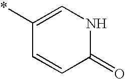 |
| 22 | 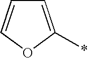 | 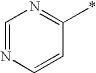 | 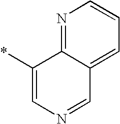 |
| 23 | 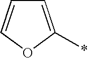 | 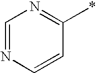 | 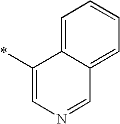 |
| 24 | 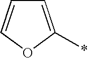 | 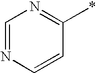 | 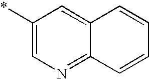 |
| 25 | 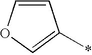 | 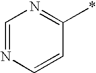 | 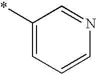 |
| 26 | 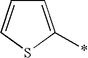 | 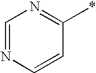 | 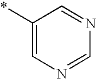 |
| 27 | 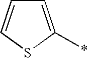 | 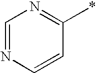 | 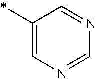 |
| 28 | 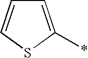 | 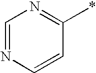 | 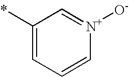 |
| 29 | 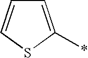 | 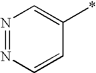 | 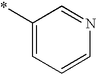 |
| 30 | 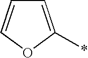 | 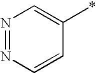 | 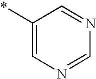 |

### Example 1

### 4′-(2-Furyl)-N-pyridin-3-yl-4,5′-bipyrimidin-2′-amine

An oven dried resealable Schlenk tube was charged with 4,5-bis(diphenylphosphino)-9,9-dimethylxanthene (Xantphos) (25.4 mg, 0.044 mmol), 3-bromopyridine (96.3 mL, 1 mmol), Cs₂CO₃ (456 mg, 1.4 mmol), 4′-(2-furyl)-4,5′-bipyrimidin-2′-amine (Preparation 1) (263 mg, 1.1 mmol) and dioxane (5 mL). The Schlenk tube was subjected to three cycles of evacuation-backfilling with argon, and tris(dibenzylidene acetone)dipalladium (0) [Pd₂(dba)₃] (18.3 mg, 0.02 mmol) was added. After three new cycles of evacuation-backfilling with argon the Schlenk tube was capped and placed in a 100° C. oil bath. After 20 h. the mixture was cooled, 10 mL of water were added and the solid was collected by filtration to give the title compound as a yellowish solid (211 mg, 67%).

m.p.: 173.6-174.4° C.

δ ¹H-NMR (DMSO-d₆): 10.26 (s, 1H), 9.24 (s, 1H), 8.98 (d, 1H), 8.84 (d, 1H), 8.69 (s, 1H), 8.33 (m, 1H), 8.22 (d, 1H), 7.76 (s, 1H), 7.55 (d, 1H), 7.39 (dd, 1H), 7.08 (d, 1H), 6.68 (m, 1H).

ESI/MS m/e: 317 ([M+H]⁺, C₁₇H₁₂N₆O).

Retention time (min.): 8

Anal. Calcd. for C₁₇H₁₂N₆O: C, 64.55; H, 3.82; N, 26.57; Found: C, 63.47; H, 3.78; N, 24.59.

Example 2

### 4′-(2-Furyl)-N-(6-methoxypyridin-3-yl)-4,5′-bipyrimidin-2′-amine

An oven dried resealable Schlenk tube was charged with CuI (18.5 mg, 0.1 mmol), 4′-(2-furyl)-4,5′-bipyrimidin-2′-amine (Preparation 1) (100 mg, 0.42 mmol), 5-bromo-2-methoxypyridine (0.065 mL, 0.5 mmol), K₂CO₃ (115 mg, 0.84 mmol) and dioxane (1 mL). After three cycles of evacuation-backfilling with argon, N,N′-dimethylethylene diamine (0.024 mL, 0.194 mmol) was added, the tube was sealed and the reaction mixture was stirred at 110° C. for 18 hours. The resulting suspension was allowed to reach room temperature and partitioned between water and dichloromethane, the organic phase was separated, washed with brine, dried (MgSO₄) and evaporated under reduced pressure. The residue was triturated with diethyl ether, the resulting solid was collected by filtration and dried under vacuum to provide the title compound as an off-white solid (100 mg, 69%).

m.p.: 212.6-213.7° C.

δ ¹H-NMR (DMSO-d⁶): 10.01 (s, 1H), 9.23 (s, 1H), 8.82 (m, 1H), 8.60 (m, 2H), 8.09 (d, 1H), 7.74 (s, 1H), 7.52 (m, 1H), 7.04 (s, 1H), 6.85 (d, 1 H), 6.67 (m, 1H), 3.84 (s, 3H).

ESI/MS m/e: 347 ([M+H]⁺, C₁₈H₁₄N₆O₂).

Retention time (min.): 12

### Example 3

### 4′-(2-Furyl)-N-pyridin-2-yl-4,5′-bipyrimidin-2′-amine

Obtained as an off-white solid (55%) from the title compound of Preparation 1 and 2-bromopyridine following the procedure of example 2.

ESI/MS m/e: 317 ([M+H]⁺, C₁₇H₁₂N₆O).

Retention time (min.): 7

### Example 4

### N-(6-Fluoropyridin-3-yl)-4′-(2-furyl)-4,5′-bipyrimidin-2′-amine

Obtained as an off-white solid (33%) from the title compound of Preparation 1 and 5-bromo-2-fluoropyridine following the procedure of example 2.

ESI/MS m/e: 335 ([M+H]⁺, C₁₇H₁₁FN₆O).

Retention time (min.): 12

### Example 5

### 4′-(2-Furyl)-N-(4-methylpyridin-3-yl)-4,5′-bipyrimidin-2′-amine

Obtained as an off-white solid (27%) from the title compound of Preparation 1 and 3-bromo-4-methylpyridine following the procedure of example 2.

ESI/MS m/e: 331 ([M+H]⁺, C₁₈H₁₄N₆O).

Retention time (min.): 7

### Example 6

### N-Pyridin-3-yl-4′-thien-2-yl-4,5′-bipyrimidin-2′-amine

Obtained as a yellowish solid (13%) from the title compound of Preparation 2 and 3-bromopyridine following the procedure of example 1.

ESI/MS m/e: 333 ([M+H]⁺, C₁₇H₁₂N₆S).

Retention time (min.): 8

### Example 7

### 4′-(3-Fluorophenyl)-N-pyridin-3-yl-4,5′-bipyrimidin-2′-amine

Obtained as a yellowish solid (22%) from the title compound of Preparation 3 and 3-bromopyridine following the procedure of example 2.

ESI/MS m/e: 345 ([M+H]⁺, C₁₉H₁₃FN₆).

Retention time (min.): 9

### Example 8

### 4′-(3-Fluorophenyl)-N-(6-methoxypyridin-3-yl)-4,5′-bipyrimidin-2′-amine

Obtained as a yellowish solid (20%) from the title compound of Preparation 3 and 5-bromo-2-methoxypyridine following the procedure of example 2.

ESI/MS m/e: 375 ([M+H]⁺, C₂₀H₁₅FN₆O).

Retention time (min.): 14

### Example 9

### 4′-(2-Furyl)-N-(6-methoxypyridin-3-yl)-24methylthio)-4,5′-bipyrimidin-2′-amine

Obtained as a yellowish solid (50%) from the title compound of Preparation 4 and 5-bromo-2-methoxypyridine following the procedure of example 2.

ESI/MS m/e: 393 ([M+H]⁺, C₁₉H₁₆N₆O₂S).

Retention time (min.): 16

### Example 10

### 4′-(3-Fluorophenyl)-2-(methylthio)-N-pyridin-3-yl-4,5′-bipyrimidin-2′-amine

Obtained as a yellowish solid (48%) from the title compound of Preparation 5 and 3-bromopyridine following the procedure of example 2.

ESI/MS m/e: 393 ([M+H]⁺, C₂₀H₁₅FN₆S).

Retention time (min.): 14

### Example 11

### 4-(2-Furyl)-5-pyridazin-4-yl-N-pyridin-3-ylpyrimidin-2-amine

Obtained as an off-white solid (15%) from the title compound of Preparation 6 and 3-bromopyridine following the procedure of example 1.

ESI/MS m/e: 317 ([M+H]⁺, C₁₇H₁₂N₆O).

Retention time (min.): 7

### Example 12

### 4′-(2-Furyl)-N-(1-oxidopyridin-3-yl)-4,5′-bipyrimidin-2′-amine

Obtained as a white solid (40%) from the title compound of Preparation 1 and 3-bromopyridine 1-oxide following the procedure of example 1.

m.p.: 206.2-206.9° C.

δ ¹H-NMR (DMSO-d₆): 10.46 (bs, 1H), 9.25 (s, 1H), 8.99 (d, 1 H), 8.85 (d, 1H), 8.72 (s, 1H), 7.91 (m, 1H), 7.74 (m, 2H), 7.58 (d, 1H), 7.39 (m, 1H), 7.07 (d, 1H), 6.69 (d, 1H).

ESI/MS m/e: 333 ([M+H]⁺, C₁₇H₁₂N₆O₂)

Retention time (min.): 8

### Example 13

### 4′-(2-Furyl)-N-pyrimidin-5-yl-4,5′-bipyrimidin-2′-amine

Obtained as a white solid (75%) from the title compound of Preparation 1 and 5-bromopyrimidine following the procedure of example 1.

m.p.: 227.8-228.9° C.

δ ¹H-NMR (DMSO-d₆): 10.41 (s, 1H), 9.26 (s, 1H), 9.26 (d, 2H), 8.85 (d, 2H), 8.72 (s, 1H), 7.77 (s, 1H), 7.56 (d, 1H), 7.03 (d, 1H), 6.68 (m, 1H).

ESI/MS m/e: 318 ([M+H]⁺, C₁₆H₁₁N₇O)

Retention time (min.): 10

### Example 14

### 4′-(2-Furyl)-N-(5-methoxypyridin-3-yl)-4,5′-bipyrimidin-2′-amine

Obtained as a yellowish solid (50%) from the title compound of Preparation 1 and 3-bromo-5-methoxypyridine following the procedure of example 1.

ESI/MS m/e: 347 ([M+H]⁺, C₁₈H₁₄N₆O₂)

Retention time (min.): 10

### Example 15

### 4′-(2-Furyl)-N-(6-methylpyridin-3-yl)-4,5′-bipyrimidin-2′-amine

Obtained as a yellow solid (50%) from the title compound of Preparation 1 and 5-bromo-2-methylpyridine following the procedure of example 1.

ESI/MS m/e: 331 ([M+H]⁺, C₁₈H₁₄N₆O).

Retention time (min.): 7

### Example 16

### 4′-(2-Furyl)-N-pyrazin-2-yl-4,5′-bipyrimidin-2′-amine

An oven dried resealable Schlenk tube was charged with 4′-(2-furyl)-4,5′-bipyrimidin-2′-amine (Preparation 1) (240 mg, 1 mmol), sodium tert-butoxide (112 mg, 1.17 mmol), 2-chloropyrazine (0.075 mL, 0.83 mmol), 2-dicyclohexylphosphino-2′-(N,N-dimethyl amino)biphenyl (33 mg, 0.083 mmol) and toluene (2 mL). The Schlenk tube was subjected to three cycles of evacuation-backfilling with argon, and palladium(II) acetate (19 mg, 0.083 mmol) was added. After three new cycles of evacuation-backfilling with argon the Schlenk tube was capped and placed in a 110° C. oil bath. After 20 h. the mixture was cooled, 10 mL of diethyl ether were added and the solid was collected by filtration to give the title compound as a white solid (88 mg, 33%)

ESI/MS m/e: 318 ([M+H]⁺, C₁₆H₁₁N₇O)

Retention time (min.): 10

### Example 17

### 5-{[4′-(2-Furyl)-4,5′-bipyrimidin-2′-yl]amino}nicotinonitrile

Obtained as an off-white solid (60%) from the title compound of Preparation 1 and 5-bromonicotinonitrile following the procedure of example 1.

ESI/MS m/e: 342 ([M+H]⁺, C₁₈H₁₁N₇O)

Retention time (min.): 12

### Example 18

### 4′-(2-Furyl)-N-(1-oxidopyrimidin-5-yl)-4,5′-bipyrimidin-2′-amine

Obtained as a white solid (70%) from the title compound of Preparation 1 and 5-bromopyrimidine 1-oxide following the procedure of example 1.

δ ¹H-NMR (DMSO-d₆): 10.67 (s, 1H), 9.26 (bs, 2H), 8.88 (d, 1H), 8.75 (bs, 2H), 8.66 (s, 1H), 7.79 (s, 1H), 7.61 (d, 1H), 7.02 (d, 1H), 6.69 (m, 1H).

ESI/MS m/e: 334 ([M+H]⁺, C₁₆H₁₁N₇O₂)

Retention time (min.): 8

### Example 19

### 4′-(2-Furyl)-N-[2-(methylthio)pyrimidin-4-yl]-4,5′-bipyrimidin-2′-amine

Obtained as an off-white solid (98%) from the title compound of Preparation 1 and 4-chloro-2-(methylthio)pyrimidine following the procedure of example 16.

δ ¹H-NMR (DMSO-d₆): 10.83 (s, 1H), 9.27 (s, 1H), 8.88 (d, 1H), 8.76 (s, 1H), 8.51 (d, 1H), 8.13 (d, 1H), 7.77 (s, 1H), 7.63 (d, 1H), 7.21 (d, 1H), 6.70 (m, 1H), 2.53 (s, 3H).

ESI/MS m/e: 364 ([M+H]⁺, C₁₇H₁₃N₇OS)

Retention time (min.): 13

### Example 20

### N-[6-(Benzyloxy)pyridin-3-yl]-4′-(2-furyl)-4,5′-bipyrimidin-2′-amine

Obtained as an off-white solid (40%) from the title compound of Preparation 1 and 2-(benzyloxy)-5-bromopyridine following the procedure of example 1.

ESI/MS m/e: 423 ([M+H]⁺, C₂₄H₁₈N₆O₂)

Retention time (min.): 16

### Example 21

### 5-{[4′-(2-Furyl)-4,5′-bipyrimidin-2′-yl]amino}pyridin-2(1H)-one

Obtained as an off-white solid (11%) by catalytic hydrogenation of the title compound of example 20 (125 mg) in tetrahydrofuran (4 mL) using 10% Palladium(II) hydroxide and a hydrogen balloon. After 48 hours the catalyst is filtered and the solvent evaporated under reduced pressure.

δ ¹H-NMR (CD₃OD): 9.24 (s, 1H), 8.76 (d, 1H), 8.60 (s, 1H), 8.26 (s, 1H), 7.82 (dd, 1H), 7.48 (m, 2H), 7.20 (d, 1H), 6.61 (m, 2H), 4.60 (bs, 1H)

ESI/MS m/e: 333 ([M+H]⁺, C₁₇H₁₂N₆O₂)

Retention time (min.): 8

### Example 22

### 4′-(2-Furyl)-N-1,6-naphthyridin-8-yl-4,5′-bipyrimidin-2′-amine

Obtained as a dark yellow solid (95%) from the title compound of Preparation 1 and 8-bromo-1,6-naphthyridine following the procedure of example 1.

ESI/MS m/e: 368 ([M+H]⁺, C₂₀H₁₃N₇O)

Retention time (min.): 11

### Example 23

### 4′-(2-Furyl)-N-isoquinolin-4-yl-4,5′-bipyrimidin-2′-amine

Obtained as a beige solid (30%) from the title compound of Preparation 1 and 4-bromoisoquinoline following the procedure of example 1.

ESI/MS m/e: 367 ([M+H]⁺, C₂₁H₁₄N₆O)

Retention time (min.): 9

### Example 24

### 4′-(2-Furyl)-N-quinolin-3-yl-4,5′-bipyrimidin-2′-amine

Obtained as a yellow solid (60%) from the title compound of Preparation 1 and 3-bromoquinoline following the procedure of example 1.

ESI/MS m/e: 367 ([M+H]⁺, C₂₁H₁₄N₆O)

Retention time (min.): 14

### Example 25

### 4′-(3-Furyl)-N-pyridin-3-yl-4,5′-bipyrimidin-2′-amine

Obtained as a beige solid (35%) from the title compound of Preparation 7 and 3-bromopyridine following the procedure of example 1.

ESI/MS m/e: 317 ([M+H]⁺, C₁₇H₁₂N₆O).

Retention time (min.): 7

### Example 26

### 4′-(3-Furyl)-N-pyrimidin-5-yl-4,5′-bipyrimidin-2′-amine

Obtained as a beige solid (42%) from the title compound of Preparation 7 and 5-bromopyrimidine following the procedure of example 1.

ESI/MS m/e: 318 ([M+H]⁺, C₁₆H₁₁N₇O)

Retention time (min.): 9

### Example 27

### N-Pyrimidin-5-yl-4′-(2-thienyl)-4,5′-bipyrimidin-2′-amine

Obtained as a yellow solid (60%) from the title compound of Preparation 2 and 5-bromopyrimidine following the procedure of example 1.

ESI/MS m/e: 334 ([M+H]⁺, C₁₆H₁₁N₇S)

Retention time (min.): 11

### Example 28

### N-(1-Oxidopyridin-3-yl)-4′-(2-thienyl)-4,5′-bipyrimidin-2′-amine

Obtained as an off-white solid (35%) from the title compound of Preparation 2 and 3-bromopyridine 1-oxide following the procedure of example 1.

ESI/MS m/e: 349 ([M+H]⁺, C₁₇H₁₂N₆OS)

Retention time (min.): 9

### Example 29

### 5-Pyridazin-4-yl-N-pyridin-3-yl-4-(2-thienyl)pyrimidin-2-amine

Obtained as a dark yellow solid (75%) from the title compound of Preparation 8 and 3-bromopyridine following the procedure of example 1.

ESI/MS m/e: 333 ([M+H]⁺, C₁₇H₁₂N₆S)

Retention time (min.): 8

### Example 30

### 4-(2-Furyl)-5-pyridazin-4-yl-N-pyrimidin-5-ylpyrimidin-2-amine

Obtained as a yellow solid (33%) from the title compound of Preparation 6 and 5-bromopyrimidine following the procedure of example 1.

ESI/MS m/e: 318 ([M+H]⁺, C₁₆H₁₁N₇O)

Retention time (min.): 9

### Composition Example 1

50,000 capsules each containing 100 mg of 4′-(2-furyl)-N-pyridin-3-yl-4,5′-bipyrimidin-2′-amine (active ingredient) were prepared according to the following formulation:

| | |
|---|---|
| Active ingredient | 5 Kg |
| Lactose monohydrate | 10 Kg |
| Colloidal silicon dioxide | 0.1 Kg |
| Corn starch | 1 Kg |
| Magnesium stearate | 0.2 Kg |

Procedure

The above ingredients were sieved through a 60 mesh sieve, and were loaded into a suitable mixer and filled into 50,000 gelatine capsules.

### Composition Example 2

50,000 tablets each containing 50 mg of 4′-(2-furyl)-N-pyridin-3-yl-4,5′-bipyrimidin-2′-amine (active ingredient) were prepared from the following formulation:

| | |
|---|---|
| Active ingredient | 2.5 Kg |
| Microcrystalline cellulose | 1.95 Kg |
| Spray dried lactose | 9.95 Kg |
| Carboxymethyl starch | 0.4 Kg |
| Sodium stearyl fumarate | 0.1 Kg |
| Colloidal silicon dioxide | 0.1 Kg |

Procedure

All the powders were passed through a screen with an aperture of 0.6 mm, then mixed in a suitable mixer for 20 minutes and compressed into 300 mg tablets using 9 mm disc and flat bevelled punches. The disintegration time of the tablets was about 3 minutes.

1. A compound of formula (I) 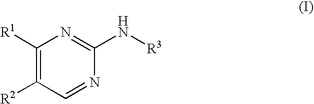
wherein
R¹ represents a monocyclic or polycyclic, aryl or heteroaryl group optionally substituted by one, two or three substituents chosen from halogen atoms, straight or branched, optionally substituted lower alkyl, hydroxy, straight or branched, optionally substituted lower alkoxy, —SH, straight or branched optionally substituted lower alkylthio, nitro, cyano, —NR′R″, —CO₂R′, —C(O)—NR′R″, —N(R′″)C(O)—R′, and —N(R′″)—C(O)NR′R″,
wherein R′, R″ and R′″ each independently represents a hydrogen atom or a straight or branched, optionally substituted lower alkyl group; or R′ and R″ together with the atom to which they are attached form a cyclic group;
R² represents a monocyclic N-containing heteroaryl group chosen from the groups of formulae (IIa) and (IIb): 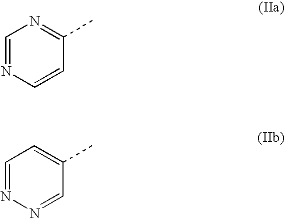
wherein the groups of formula (IIa) and (IIb) are each independently optionally substituted by one, two or three substituents chosen from halogen atoms, straight or branched, optionally substituted lower alkyl, hydroxy, straight or branched, optionally substituted lower alkoxy, —SH, straight or branched optionally substituted lower alkylthio, nitro, cyano, —NR′R″, —CO₂R′, —C(O)—NR′R″, —N(R′″)C(O)—R′, and —N(R′″)—C(O)NR′R″,
wherein R′, R″ and R′″ each independently represents a hydrogen atom or a straight or branched, optionally substituted lower alkyl group; or R′ and R″ together with the atom to which they are attached form a cyclic group.
R³ represents a monocyclic or polycyclic, heteroaryl group being optionally substituted by one, two or three substituents chosen from halogen atoms, straight or branched, optionally substituted lower alkyl, hydroxy, oxo, straight or branched, optionally substituted lower alkoxy, —SH, straight or branched optionally substituted lower alkylthio, nitro, cyano, —NR′R″, —CO₂R′, —C(O)—NR′R″, —N(R′″)C(O)—R′, and —N(R′″)—C(O)NR′R″,
wherein R′, R″ and R′″ each independently represents a hydrogen atom or a straight or branched, optionally substituted lower alkyl group; or R′ and R″ together with the atom to which they are attached form a cyclic group;
or an N-oxide thereof or a pharmaceutically acceptable salt thereof;

2. A compound according to claim 1, wherein R³ represents either
a monocyclic or polycyclic heteroaryl group comprising a nitrogen-containing six-membered ring; or
a monocyclic five-membered heteroaryl group not containing nitrogen in the ring structure,
wherein each heteroaryl group is independently optionally substituted by one, two or three substituents chosen from halogen atoms, straight or branched, optionally substituted lower alkyl, hydroxy, oxo, straight or branched, optionally substituted lower alkoxy, —SH, straight or branched optionally substituted lower alkylthio, nitro, cyano, —NR′R″, —CO₂R′, —C(O)—NR′R″, —N(R′″)C(O)—R′, and —N(R′″)—C(O)NR′R″,
wherein R′, R″ and R′″ each independently represents a hydrogen atom or a straight or branched, optionally substituted lower alkyl group; or R′ and R″ together with the atom to which they are attached form a cyclic group.

3. A compound according to claim 2, wherein R³ represents a monocyclic or polycyclic heteroaryl group comprising a nitrogen-containing six-membered ring,
wherein the heteroaryl group is optionally substituted by one, two or three substituents chosen from halogen atoms, straight or branched, optionally substituted lower alkyl, hydroxy, oxo, straight or branched, optionally substituted lower alkoxy, —SH, straight or branched optionally substituted lower alkylthio, nitro, cyano, —NR′R″, —CO₂R′, —C(O)—NR′R″, —N(R′″)C(O)—R′, and —N(R′″)—C(O)NR′R″,
wherein R′, R″ and R′″ each independently represents a hydrogen atom or a straight or branched, optionally substituted lower alkyl group; or R′ and R″ together with the atom to which they are attached form a cyclic group.

4. A compound according to claim 1, wherein R³ is chosen from pyridine, pyrimidine, pyridazine, isoquinoline, quinoline, naphthyridine, pyridine-2(1H)-one, furan and thiophene; each of them optionally substituted by one, two or three substituents chosen from halogen atoms, straight or branched, optionally substituted lower alkyl, hydroxy, oxo, straight or branched, optionally substituted lower alkoxy, —SH, straight or branched optionally substituted lower alkylthio, nitro, cyano, —NR′R″, —CO₂R′, —C(O)—NR′R″, —N(R′″)C(O)—R′, and —N(R′″)—C(O)NR′R″,
wherein R′, R″ and R′″ each independently represents a hydrogen atom or a straight or branched, optionally substituted lower alkyl group; or R′ and R″ together with the atom to which they are attached form a cyclic group.

5. A compound according to claim 4, wherein R³ is chosen from pyridine and pyridine-2(1H)-one, each of them optionally substituted by one, two or three substituents chosen from halogen atoms, straight or branched, optionally substituted lower alkyl, hydroxy, oxo, straight or branched, optionally substituted lower alkoxy, —SH, straight or branched optionally substituted lower alkylthio, nitro, cyano, —NR′R″, —CO₂R′, —C(O)—NR′R″, —N(R′″)C(O)—R′, and —N(R′″)—C(O)NR′R″,
wherein R′, R″ and R′″ each independently represents a hydrogen atom or a straight or branched, optionally substituted lower alkyl group; or R′ and R″ together with the atom to which they are attached form a cyclic group.

6. A compound according to claim 1, wherein R³ is chosen from pyridine, pyrimidine, pyridazine, isoquinoline, quinoline, naphthyridine and pyridine-2(1H)-one, each of them optionally substituted by a substituent chosen from halogen atoms, straight or branched, optionally substituted lower alkyl, oxo, straight or branched, optionally substituted lower alkoxy, straight or branched optionally substituted lower alkylthio and cyano groups.

7. A compound according to claim 1, wherein R³ is chosen from pyridine and pyridine-2(1H)-one, each of them optionally substituted by a substituent chosen from halogen atoms, straight or branched, optionally substituted lower alkyl, oxo, straight or branched, optionally substituted lower alkoxy, straight or branched optionally substituted lower alkylthio and cyano groups.

8. A compound according to claim 1, wherein R¹ represents a group chosen from phenyl, furan-2-yl, furan-3-yl, thien-2-yl, thien-3-yl, pyridin-2-yl, pyridin-3-yl and pyridin-4-yl, each of them optionally substituted by one, two or three substituents chosen from halogen atoms, straight or branched, optionally substituted lower alkyl, hydroxy, straight or branched, optionally substituted lower alkoxy, —SH, straight or branched optionally substituted lower alkylthio, nitro, cyano, —NR′R″, —CO₂R′, —C(O)—NR′R″, —N(R′″)C(O)—R′, —N(R′″)—C(O)NR′R″,
wherein R′, R″ and R′″ each independently represents a hydrogen atom or a straight or branched, optionally substituted lower alkyl group; or R′ and R″ together with the atom to which they are attached form a cyclic group.

9. A compound according to claim 8, wherein R¹ represents a group chosen from phenyl, furan-2-yl, furan-3-yl and thien-2-yl, each of them optionally substituted by an halogen atom.

10. A compound according to claim 9, wherein R¹ represents a group chosen from unsubstituted furan-2-yl and unsubstituted thien-2-yl.

11. A compound according to claim 1, wherein R² represents a pyrimidinyl or pyridazinyl group; wherein the pyrimidinyl or pyridazinyl group is optionally substituted by one, two or three substituents chosen from halogen atoms, straight or branched, optionally substituted lower alkyl, hydroxy, straight or branched, optionally substituted lower alkoxy, —SH, straight or branched optionally substituted lower alkylthio, nitro, cyano, —NR′R″, —CO₂R′, —C(O)—NR′R″, —N(R′″)C(O)—R′, _N(R′″)—C(O)NR′R″,
wherein R′, R″ and R′″ each independently represents a hydrogen atom or a straight or branched, optionally substituted lower alkyl group; or R′ and R″ together with the atom to which they are attached form a cyclic group.

12. A compound according to claim 11, wherein R² represents a pyrimidinyl or pyridazinyl group; wherein the pyrimidinyl or pyridazinyl group is optionally substituted by a straight or branched, optionally substituted lower alkylthio group.

13. A compound according to claim 12, wherein R² represents an unsubstituted pyrimidin-4-yl or an unsubstituted pyridazin-4-yl group.

14. A compound according to claim 13, wherein R¹ represents a group chosen from unsubstituted furan-2-yl and unsubstituted thien-2-yl, R² represents an unsubstituted pyrimidin-4-yl or an unsubstituted pyridazin-4-yl and wherein R³ is chosen from the group consisting of pyridine, pyrimidine, pyridazine, isoquinoline, quinoline, naphthyridine and pyridine-2(1H)-one, all of them optionally substituted by a substituent chosen from the group consisting of halogen atoms, straight or branched, optionally substituted lower alkyl, oxo, straight or branched, optionally substituted lower alkoxy, straight or branched optionally substituted lower alkylthio and cyano groups.

15. A compound according to claim 1, chosen from:
4′-(2-furyl)-N-pyridin-3-yl-4,5′-bipyrimidin-2′-amine
4′-(2-furyl)-N-(6-methoxypyridin-3-yl)-4,5′-bipyrimidin-2′-amine
4′-(2-furyl)-N-pyridin-2-yl-4,5′-bipyrimidin-2′-amine
N-(6-fluoropyridin-3-yl)-4′-(2-furyl)-4,5′-bipyrimidin-2′-amine
4′-(2-furyl)-N-(4-methylpyridin-3-yl)-4,5′-bipyrimidin-2′-amine
N-pyridin-3-yl-4′-thien-2-yl-4,5′-bipyrimidin-2′-amine
4′-(3-fluorophenyl)-N-pyridin-3-yl-4,5′-bipyrimidin-2′-amine
4′-(3-fluorophenyl)-N-(6-methoxypyridin-3-yl)-4,5′-bipyrimidin-2′-amine
4′-(2-furyl)-N-(6-methoxypyridin-3-yl)-2-(methylthio)-4,5′-bipyrimidin-2′-amine
4′-(3-fluorophenyl)-2-(methylthio)-N-pyridin-3-yl-4,5′-bipyrimidin-2′-amine
4-(2-furyl)-5-pyridazin-4-yl-N-pyridin-3-ylpyrimidin-2-amine
4′-(2-furyl)-N-(1-oxidopyridin-3-yl)-4,5′-bipyrimidin-2′-amine
4′-(2-furyl)-N-pyrimidin-5-yl-4,5′-bipyrimidin-2′-amine
4′-(2-furyl)-N-(5-methoxypyridin-3-yl)-4,5′-bipyrimidin-2′-amine
4′-(2-furyl)-N-(6-methylpyridin-3-yl)-4,5′-bipyrimidin-2′-amine
4′-(2-furyl)-N-pyrazin-2-yl-4,5′-bipyrimidin-2′-amine
5-{[4′-(2-furyl)-4,5′-bipyrimidin-2′-yl]amino}nicotinonitrile
4′-(2-furyl)-N-(1-oxidopyrimidin-5-yl)-4,5′-bipyrimidin-2′-amine
4′-(2-furyl)-N-[2-(methylthio)pyrimidin-4-yl]-4,5′-bipyrimidin-2′-amine
N-[6-(benzyloxy)pyridin-3-yl]-4′-(2-furyl)-4,5′-bipyrimidin-2′-amine
5-{[4′-(2-furyl)-4,5′-bipyrimidin-2′-yl]amino}pyridin-2(1H)-one
4′-(2-furyl)-N-1,6-naphthyridin-8-yl-4,5′-bipyrimidin-2′-amine
4′-(2-furyl)-N-isoquinolin-4-yl-4,5′-bipyrimidin-2′-amine
4′-(2-furyl)-N-quinolin-3-yl-4,5′-bipyrimidin-2′-amine
4′-(3-furyl)-N-pyridin-3-yl-4,5′-bipyrimidin-2′-amine
4′-(3-furyl)-N-pyrimidin-5-yl-4,5′-bipyrimidin-2′-amine
N-pyrimidin-5-yl-4′-(2-thienyl)-4,5′-bipyrimidin-2′-amine
N-(1-oxidopyridin-3-yl)-4′-(2-thienyl)-4,5′-bipyrimidin-2′-amine
5-pyridazin-4-yl-N-pyridin-3-yl-4-(2-thienyl)pyrimidin-2-amine; and
4-(2-furyl)-5-pyridazin-4-yl-N-pyrimidin-5-ylpyrimidin-2-amine.

16. A process for producing a compound as claimed in claim 1, wherein a compound of formula (IX) is coupled with a compound of formula (III); wherein and X is a halogen atom, 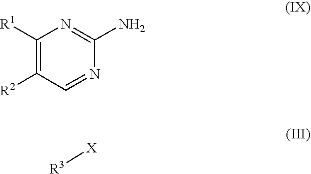
and optionally converting the resulting compound into an N-oxide thereof or a pharmaceutically acceptable salt thereof.

17. (canceled)

18. A pharmaceutical composition comprising a compound as claimed in claim 1 and a pharmaceutically acceptable diluent or carrier.

19. (canceled)

20. (canceled)

21. A method for treating a subject afflicted with a pathological condition or disease susceptible to amelioration by antagonism of the A_{2B} adenosine receptor, comprising administering to said subject an effective amount of a compound as claimed in claim 1.

22. A method according to claim 21, wherein the pathological condition or disease is chosen from asthma, bronchoconstriction, allergic diseases, hypertension, atherosclerosis, reperfusion injury, myocardial ischemia, retinopathy, inflammation, gastrointestinal tract disorders, cell proliferation disorders, diabetes mellitus, and autoimmune diseases.

23. A process according to claim 16, wherein the halogen atom is chosen from bromine, iodine and chlorine.
